(19) United States
(12) Patent Application Publication
(10) Pub. No.: US 2006/0122565 A1
(43) Pub. Date: Jun. 8, 2006

(54) Switch structures or the like based on a thermoresponsive polymer

(76) Inventor: Chee Choong Kooi, Glugor (MY)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/141,730

(22) Filed: May 31, 2005

(30) Foreign Application Priority Data
Nov. 23, 2004 (MY) PI 20044842

(51) Int. Cl. A61M 5/00

(52) U.S. Cl. 604/246; 137/554

(57) ABSTRACT

Briefly, in accordance with one embodiment of the invention, a switch structure or the like such as a valve, motor, or optical switch, may be constructed based on a thermoresponsive polymer. At a first temperature the thermoresponsive polymer may be in a first volume state, and at a second temperate the thermoresponsive polymer may be in a second volume state. The change in volume of the thermoresponsive polymer may be operative to push or pull the mechanical structures of the switch, valve, motor, optical switch, and so on, to effectuate operation of the structures.

### BACKGROUND OF THE INVENTION

Microelectromechanical systems (MEMS) are micro-scale devices that combine both mechanical and electrical features. Such MEMS devices may include, for example, switches, filters, resonators, movable mirrors, or the like with typical applications in communications devices and systems where MEMS devices and structures may be utilized in electronic devices such as cellular telephones and other radio systems, or switches and routers used for network systems. Ever increasingly, MEMS technology is being applied to biological systems in a field of technology that generally may be referred to as BioMEMS where MEMS devices and technology may be utilized in a variety of applications in biology, medicine, biological research, mircofluidicis, or the like.

### DESCRIPTION OF THE DRAWING FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic diagram of a switch based on the operation of a thermoresponsive polymer in accordance with one or more embodiments of the present invention;

FIG. 2 is a schematic diagram of the physical changes undergone by a thermoresponsive polymer in response to a thermal stimulus in accordance with one or more embodiments of the present invention;

FIG. 3 is a graph illustrating the change in volume that a thermoresponsive polymer may exhibit as a function of temperature in accordance with one or more embodiments of the present invention;

FIG. 4 is a schematic illustration of example thermoresponsive polymers suitable for utilization in a switch structure or the like in accordance with one or more embodiments of the present invention;

FIG. 5 is a schematic illustration of the synthesis of a thermoresponsive polymer gel suitable for utilization in a switch structure or the like in accordance with one or more embodiments of the present invention;

FIG. 6 is a schematic illustration of a method for constructing a switch structure or the like based on a thermoresponsive polymer in accordance with one or more embodiments of the present invention;

FIG. 7 is a schematic diagram of a heating actuator to control a thermoresponsive polymer in accordance with one or more embodiments of the present invention;

FIG. 8 is a schematic diagram illustrating alternative structures that may be actuated or controlled based on a thermoresponsive polymer in accordance with one or more embodiments of the present invention; and

FIG. 9 is a schematic diagram illustrating a system for controlling delivery of a substance to a mammal where the system may include a thermoresponsive polymer based valve in accordance with one or more embodiments of the present invention.

It will be appreciated that for simplicity and clarity of illustration, elements illustrated in the figures have not necessarily been drawn to scale. For example, the dimensions of some of the elements are exaggerated relative to other elements for clarity. Further, where considered appropriate, reference numerals have been repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail.

In the following description and claims, the terms coupled and connected, along with their derivatives, may be used. In particular embodiments, connected may be used to indicate that two or more elements are in direct physical or electrical contact with each other. Coupled may mean that two or more elements are in direct physical or electrical contact. However, coupled may also mean that two or more elements may not be in direct contact with each other, but yet may still cooperate or interact with each other.

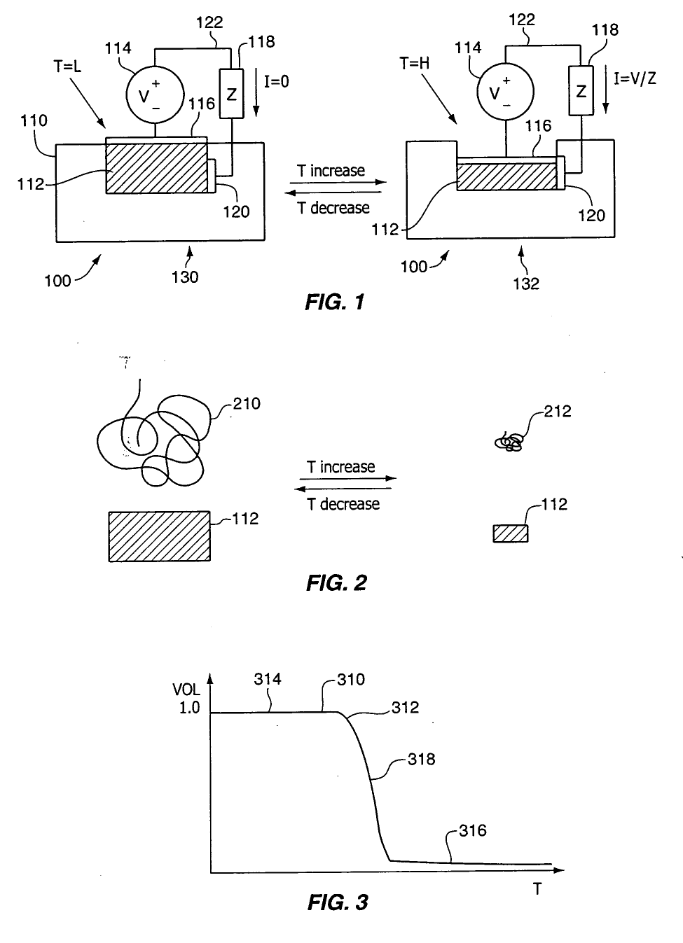

Referring now to FIG. 1, a schematic diagram of a switch based on the operation of a thermoresponsive polymer in accordance with one or more embodiments of the present invention will be discussed. As shown in FIG. 1, a switch 100 may be constructed using a thermoresponsive polymer 112 disposed on or in a substrate 110. In one embodiment of the invention, substrate 110 may be a semiconductor material such as silicon (Si), although the scope of the invention is not limited in this respect. Switch 100 may be in an open state 130 as follows. A voltage source 114 may apply a voltage to a load 118. In an open state 130, thermoresponsive polymer 130 may have a conductor 116 disposed on a surface of conductor 116. When a temperature, T having a lower value, is applied to thermoresponsive polymer 112, for example the ambient temperature or an applied or detected thermostimulus, thermoresponsive polymer 112 may be in an expanded volume state wherein conductor 116 does not contact conductor 120 that is coupled to load 118. In such an arrangement, a circuit 122 may be in an open circuit state wherein no current flows through load 118 from voltage source 114. When circuit 122 is in an open circuit state, the current I will have a zero or nearly zero value.

As the value of temperature T is increased, when T has a sufficiently high value, thermoresponsive polymer 122 may change to a contracted volume state wherein conductor 116 may contact conductor 120 to complete a circuit so that circuit 122 is in a closed circuit state. As a result, switch 130 may be considered to have changed from an open state 130 to a closed state 132 as shown in FIG. 1 in response to the temperature value T which operates to actuate switch 100. In the closed state 132, current may flow through circuit 122 wherein the value of the current I may be the value of the voltage V applied by voltage source 114 divided by the value of the impedance Z of the load 188 (I=V/Z), although the scope of the invention is not limited in this respect. Likewise, when the temperature T decreases to a sufficiently lower value, thermoresponsive polymer 112 expands to an expanded volume state wherein conductor 116 no longer contacts conductor 120 and the circuit 122 becomes an open circuit. In such an arrangement, switch 100 may be actuated in response to a thermal stimulus, although the scope of the invention is not limited in this respect.

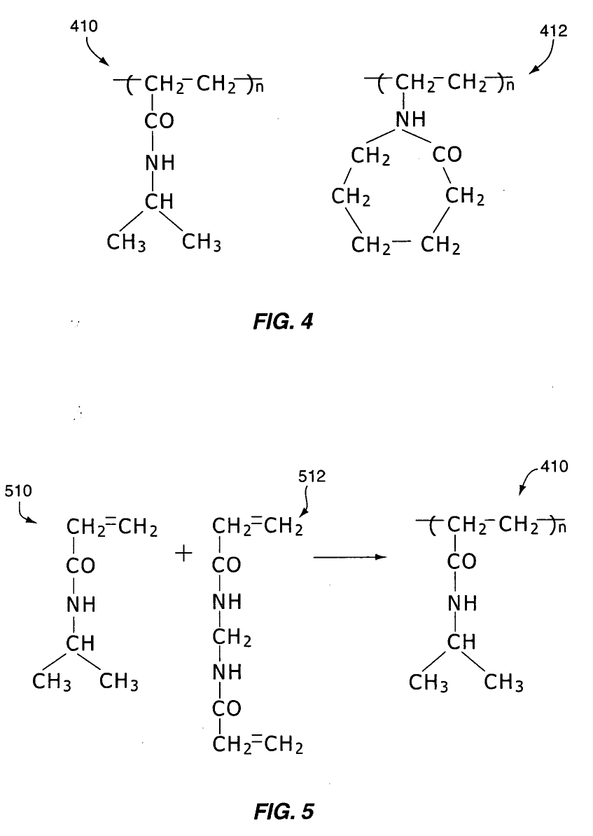

Referring now to FIG. 2, a schematic diagram of the physical changes undergone by a thermoresponsive polymer in response to a thermal stimulus in accordance with one or more embodiments of the present invention will be discussed. Thermoresponsive polymer 112 may be considered as a type of smart polymer that is able to react in response to thermal stimulus. Polymers with such properties include but are not limited to poly(N-isopropylacrylamide) and poly(N-vinylcaprolactam). Such polymers are polymers which may contain both polar and non-polar pendant groups. The chemical structures of such example polymers are shown in FIG. 4. The solubility of these polymers may be dependent on the temperature of the solution since the polymer is not thermodynamically stable in solution based on the Gibbs free energy (ΔG) equation:
ΔG=ΔH−TΔS
where ΔH is the change in enthalpy, T is the temperature, and ΔS is the change in entropy. In a such a polymer solution, ΔH may both be ΔS lower valued and negatively valued. At a lower temperature (lower T value), the enthalpy may be larger in magnitude than the entropy and hence, the ΔG is negative which means that the reaction is feasible and the polymer is soluble. On the other hand, at higher temperature (larger T value), the entropy portion may be larger than that of the enthalpy resulting in a positive ΔG value. Hence, at higher temperatures, the reaction is not feasible meaning that the polymer has a reduced solubility. The temperature that the polymer starts to react to a thermal stimulus is called the lower critical solution temperature (LCST). The solubility phenomenon based on temperature may be a reversible process, although the scope of the invention is not limited in this respect.

As shown in FIG. 2, in terms of physical changes thermoresponsive polymer 112 may change its conformation at the LCST from a soluble flexible random coil 210 having a higher solubility at a lower temperature, a temperature value less than LCST, to a more tightly coiled globular structure 212 having a lower solubility at higher temperature, a temperature value greater than the LCST. In the case of where thermoresponsive polymer 112 is in a gel form, the gel may expand to a higher volume state at a lower temperature and may contract to a lower volume state at a higher temperature when the temperature is greater than its LCST, although the scope of the invention is not limited in this respect. Thus, the thermoresponsive behavior of the polymer may be due to the variance in thermodynamic stability of the polymer in water at different temperatures. At a lower temperature, thermoresponsive polymer 112 may be thermodynamically stable in solution and has an expanded coil conformation 210. When heated to higher temperature, thermoresponsive polymer 112 may not be thermodynamically stable in solution, and a stable conformation may be a contracted coiled structure 212.

Referring now to FIG. 3, a graph illustrating the change in volume that a thermoresponsive polymer may exhibit as a function of temperature in accordance with one or more embodiments of the present invention will be discussed. The graph of FIG. 3 shows a plot 310 of the volume of thermoresponsive polymer 112 such as a gel form of thermoresponsive polymer 112 with respect to temperature. Temperature is represented on the abscissa axis and volume is represented on the ordinate axis. The LCST value may occur at a point 312. When the temperature is lower than the LCST, the volume of thermoresponsive gel 112 remains relatively constant at an expanded volume level normalized to 1.0 volume units. As the temperature is increased to and beyond the LCST value, the volume of thermoresponsive polymer 112 drops at a relatively rapid rate having a slope as indicated at 318. At a temperature slightly greater than the LCST value, the volume of thermoresponsive polymer 112 falls to a value much lower than full volume at point 316, where the volume of thermoresponsive polymer remains relative constant where the temperature is greater than the LCST value. Thus, as shown in FIG. 3, the thermoresponsive behavior of thermoresponsive polymer 112 may be analogous to the behavior of a switch or the like actuator where there may be a discrete change in value in the transition from a lower temperature state to a higher temperature state, and likewise in the transition from a higher temperature state to a lower temperature state, with a relatively rapid change from one state to the other, although the scope of the invention is not limited in this respect.

Referring now to FIG. 4, a schematic illustration of example thermoresponsive polymers suitable for utilization in a switch structure or the like in accordance with one or more embodiments of the present invention will be discussed. In one embodiment of the invention, thermoresponsive polymer 112 may be a poly(N-ispropylacrylamide) polymer 410 (poly NIPAM) having a chemical structure as shown. In an alternative embodiment of the invention, thermoresponsive polymer may be a poly(N-vinylcaprolactam) polymer 412 (poly NVCap) having a chemical structure as shown.

Referring now to FIG. 5, a schematic illustration of the synthesis of a thermoresponsive polymer gel suitable for utilization in a switch structure or the like in accordance with one or more embodiments of the present invention will be discussed. A gel network may be obtained by utilizing cross-linking agents such as bisacrylamide 512 or alternatively divinyl benzene (not shown) to polymerize N-isopropylacrylamide monomers 510 as shown in FIG. 5. The resulting cross-linked polymer gel networks comprising cross-linked poly(N-isopropylacrylamide) 410 may be sufficiently mechanically strong to pull, push or otherwise actuate parts when under contraction or expansion.

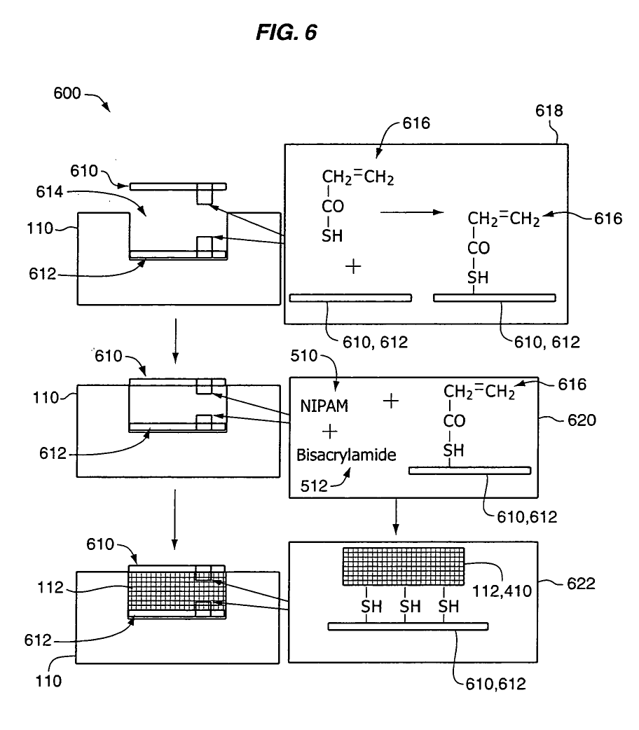

Referring now to FIG. 6, a schematic illustration of a method for constructing a switch structure or the like based on a thermoresponsive polymer in accordance with one or more embodiments of the present invention will be discussed. As shown in FIG. 6, the method 600 may generally include the following. A well or trench 614 may be plated with a metal to form a base plate 612 in the well 614. The metal used to form base plate 612 may be for example gold (Au) to allow mercapto functional groups (SH) to easily couple to base plate 612, although the scope of the invention is not limited in this respect. Likewise, a metal top plate 610 may be formed from a metal such as gold. Base plate 612 and top plate 610 may be grafted with mercaptoacetic acid 616 as shown in box 618. As show in box 620, the NIPAM or NVCap monomers such as NIPAM 510 and a cross-linker such as bisacrylamide 512 may be placed in well 614, and then top plate 610 may be positioned to seal well 614. Free radical polymerization may be carried out thermally or photochemically, for example. The mercapto group (SH) may directly couple with the metal of base plate 612 or top plate 610 making the metal surface functionalized with vinyl groups which may react with NIPAM 410 or NVCap 412 monomers such as NIPAM monomer 510 during polymerization to form thermoresponsive polymer 112 in gel form, which may enable thermoresponsive polymer 112 gel to covalently bond to the metal surface of top plate 610 or base plate 612 with a stronger adhesion, as shown in box 622. The polymerized thermoresponsive polymer 112 gel in well 614 may then be utilized in a switch structure or the like device in accordance with the present invention, although the scope of the invention is not limited in this respect.

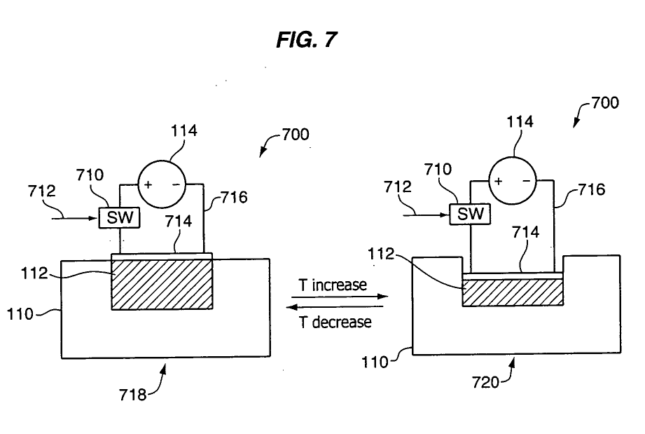

Referring now to FIG. 7, is a schematic diagram of a heating actuator to control a thermoresponsive polymer in accordance with one or more embodiments of the present invention will be discussed. In one embodiment of the invention, a heating actuator 700 may be constructed along with thermoresponsive polymer 112. A voltage source 114 may couple to a heating element 714 as a load to voltage source 114 in circuit 716. A switch 710 may be coupled in circuit 716 to selectively open or close circuit 716. Switch 710 may be, for example, a mechanical switch or an electronic switch, although the scope of the invention is not limited in this respect. Actuation of switch 710 may be controlled via a control signal 712 that causes switch 710 to open or close in response to control signal 712. In an open state 718, switch 710 may be opened and circuit 716 may be an open circuit. In such an arrangement, little or no current may flow from voltage source 114 to heating element 714. As a result, thermoresponsive polymer 112 may be at a lower temperature, and as a result may be at a higher volume state. In a close state 720, switch 710 may be closed and circuit 716 may be a closed circuit. In such an arrangement, current may flow from voltage source 114 through heating element 714 which may cause an increase in the temperature of thermoresponsive polymer 112. When thermoresponsive polymer 112 is heated to a temperature at or beyond the LCST, thermoresponsive polymer 112 may contract to a lower volume state. Likewise, when switch 710 is moved from a closed state to an open state, little or no current may flow through heating element 714, wherein thermoresponsive polymer 112 may transition from a lower volume state to a higher volume state when the temperature of thermoresponsive polymer falls below the LCST. In such a heating actuator 700, switch structures or the like may be constructed based on the temperature controlled heating and cooling of thermoresponsive polymer 112 via heating actuator 700, although the scope of the invention is not limited in this respect.

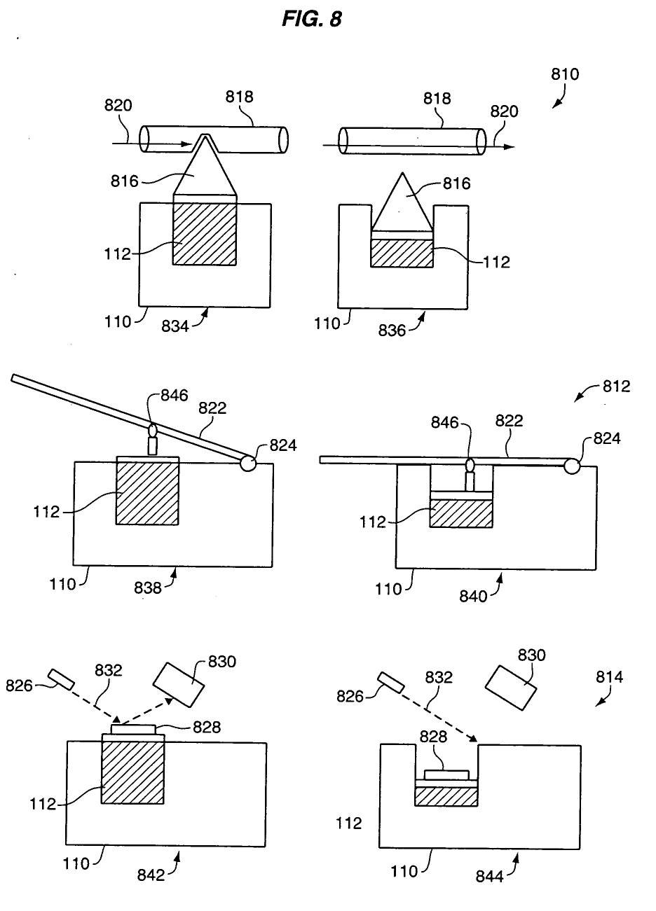

Referring now to FIG. 8, a schematic diagram illustrating alternative structures that may be actuated or controlled based on a thermoresponsive polymer in accordance with one or more embodiments of the present invention will be discussed. As shown in FIG. 8, thermoresponsive polymer 112 gels in accordance with the present invention may be sufficiently mechanically strong and may be utilized to move parts and other structures via temperature controlled contraction and expansion. When thermoresponsive polymer 112 contracts, a pulling action may be provided, and when thermoresponsive polymer expands, a pushing action may be provided. In accordance with one or more embodiments of the invention, an expansion or pushing may occur at lower temperatures, and a contraction or pulling may occur at higher temperatures above the LCST. Thus, the structures shown in FIG. 8 may be constructed as a valve 810 to control fluid flow, as a motor 812 to move a cantilever or rotor, or as an optical switch 814 to move a mirror. Being thermoresponsive, such structures may be operated as a temperature sensor or switch to control the operation thereof, although the scope of the invention is not limited in this respect.

As shown in FIG. 8, valve 810 may include a wedge 816 or a structure having a similar function may be couple to a tube 818 to operate as a valve to control the flow a fluid 820 through tube 818. The fluid 820 may be, for example, a liquid or a gas, or a mixture of solid particulates having a size to allow fluid like flow through tube 818, although the scope of the invention is not limited in this respect. In one embodiment, tube 818 may be constructed from a flexible material wherein thermoresponsive polymer 112 may be in an expanded volume state 834 when its temperature is lower than the LCST, thereby causing wedge to pinch tube 818 and restrict or stop the flow of fluid 820 there through. When thermoresponsive polymer 112 is heated to a temperature greater than the LCST, thermoresponsive polymer 112 may be in a lower volume state 826, thereby causing wedge to retreat from tube 818 and allowing fluid 820 to flow through tube 818, although the scope of the invention is not limited in this respect.

A motor 812 may include a cantilevered member 822 coupled to a pivot 824 where the cantilevered member 822 may be coupled to thermoresponsive polymer 112 at a point 846 distal from pivot 824. When the temperature of thermoresponsive polymer 112 is lower than the LCST, thermoresponsive polymer 112 may be in a higher volume state 838, and the cantilevered member 822 may be disposed at a first angle with respect to substrate 112. When the temperature of thermoresponsive polymer 112 is greater than the LCST, thermoresponsive polymer 112 may be in a lower volume state 840, thereby moving cantilevered member 822 to be disposed at a second angle with respect to substrate 112. Likewise, when the temperature of falls below the LCST, thermoresponsive polymer 112 may transition from lower volume state 840 to higher volume state 838 thereby changing the position of cantilevered member, although the scope of the invention is not limited in this respect.

An optical switch 814 may include a mirror 828 or similar reflective surface disposed on thermoresponsive polymer 112. When the temperature of thermoresponsive polymer is lower than the LCST, thermoresponsive polymer 112 may be in an expanded volume state 842, thereby positioning mirror in a first position. In the first position, a light source 826 such as a laser light source or a vertical cavity surface emitting laser (VCSEL) may emit a ray of light 832 that may impinge upon mirror 828 and be reflected to and detected by a light detector 830 which may be for example a charge coupled device (CCD), a complementary metal oxide semiconductor (CMOS) detector, a p-type-intrinsic-n-type (PIN) diode, or similar. When the temperature of thermoresponsive polymer is greater than the LCST, thermoresponsive polymer 112 may be in a lower volume state 844, thereby positioning mirror 828 in a second position. In the second position, the ray of light 832 emitted from light source 826 may not impinge upon mirror 828 and therefore not be reflected to and detected by detector 830, although the scope of the invention is not limited in this respect.

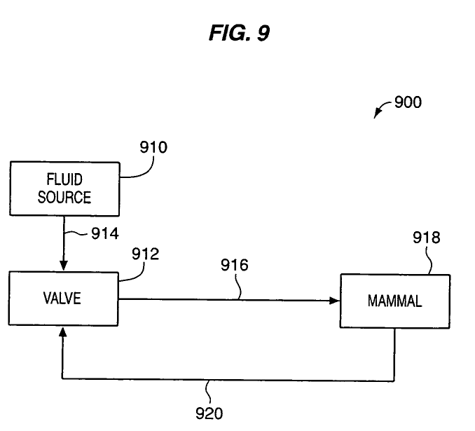

Referring now to FIG. 9, a schematic diagram illustrating a system for controlling delivery of a substance to a mammal where the system may include a thermoresponsive base syringe in accordance with one or more embodiments of the present invention will be discussed. As shown in FIG. 9, delivery system 900 may include a fluid source 910 containing a fluid that may be desired to be administered to a mammal 918. In one embodiment of the invention, mammal 918 may be an animal such as a canine or a feline mammal, and in an alternative embodiment of the invention, mammal 918 may be a human, although the scope of the invention is not limited in this respect. The fluid from fluid source 910 may be a liquid or a gas, or may be composed of particulate matter or powder, although the scope of the invention is not limited in this respect. In one embodiment of the invention, the fluid may be a liquid such as blood or blood plasma, or the fluid may be an intravenous solution containing, for example, glucose, potassium, or other minerals or nutrients, although the scope of the invention is not limited in this respect. In one particular embodiment of the invention, the fluid may be a drug or similar chemical administered to mammal 918, for example insulin or potossin, although the scope of the invention is not limited in this respect.

During operation of delivery system 900, the fluid from fluid source 910 may be routed through valve 912, which may be for example valve 810 in FIG. 8, and routed to mammal 918 via administration conduit 916. Administration conduit 916 may be for example a flexible tube or syringe although the scope of the invention is not limited in this respect. A feedback signal 920 may be provided from mammal 918 to valve 912 to control or regulate the administration of the fluid from fluid source 910 to mammal 918. In one embodiment of the invention, feedback signal 920 may include a temperature stimulus generated by the body temperature of mammal 918 to modulate operation of valve 912 which operates based upon the action of thermoresponsive polymer and thereby modulates the flow of the fluid from fluid source 910 to mammal 918 according to the value of the temperature stimulus. In one embodiment of the invention, valve may be disposed on the body of mammal 918 or internal to the body of mammal 918 to detect the body temperature of mammal 918. In an alternative embodiment of the invention, feedback signal 920 may be an electrical signal generated by a sensor (not shown) that may be responsive to a state of mammal 918 such as temperature, oxygen concentration, or other chemical level or concentration, although the scope of the invention is not limited in this respect. In such an embodiment, the electrical signal may operate as a control signal 712 to control a heating element 714 of valve 912 to operate valve 912 based on heating system 700 as shown in and described with respect to FIG. 7, although the scope of the invention is not limited in this respect.

As shown by example with delivery system 900 of FIG. 9, a thermoresponsive valve 912 may be constructed based upon a thermoresponsive polymer in accordance with the present invention to operate as a temperature switch or a temperature sensor sensors since thermoresponsive polymer may be designed to operate at a selected LCST temperature. For example, poly(N-isopropylacrylamide) 410 may be particularly suitable for biological based systems such as delivery system 900 since the nominal LCST of poly(N-isopropylacrylamide) 410 is at or near 32° C. which is sufficiently close to a normal human body temperature of 37° C. Furthermore, the LCST may be modified higher or lower to be suitable for a desired application. The modification of the LCST of thermoresponsive polymer 112 may be accomplished, for example, via copolymerization of hydrophobic compounds with the base polymer to shift the LCST to a lower level, or via polymerization of hydrophilic compounds with the base polymer to shift the LCST to a higher level, for example from 32° C. to 37° C., although the scope of the invention is not limited in this respect.

Although the invention has been described with a certain degree of particularity, it should be recognized that elements thereof may be altered by persons skilled in the art without departing from the spirit and scope of the invention. It is believed that the switch structures or the like based on a thermoresponsive polymer of the present invention and many of its attendant advantages will be understood by the forgoing description, and it will be apparent that various changes may be made in the form, construction and arrangement of the components thereof without departing from the scope and spirit of the invention or without sacrificing all of its material advantages, the form herein before described being merely an explanatory embodiment thereof, and further without providing substantial change thereto. It is the intention of the claims to encompass and include such changes.

What is claimed is:

1. An apparatus, comprising:
a substrate having a well formed thereon; and
a thermoresponsive polymer disposed in the well, the thermoresponsive polymer having a first contact and the substrate having a second contact disposed proximate to the thermoresponsive polymer;
wherein the thermoresponsive polymer has an expanded volume at a lower temperature and the first contact does not couple to the second contact, and wherein the thermoresponsive polymer has a contracted volume at a higher temperature to cause the first contact to couple to the second contact.

2. An apparatus as claimed in claim 1, wherein the thermoresponsive polymer is in a gel form.

3. An apparatus switch as claimed in claim 1, wherein the thermoresponsive polymer comprises at least one of poly(N-isopropylacrylamide) or poly(N-vinylcaprolactam).

4. An apparatus switch as claimed in claim 1, wherein the lower temperature is less than a lower critical solution temperature of the thermoresponsive polymer and the higher temperature is greater than the lower critical solution temperature of the thermoresponsive polymer

5. An apparatus, comprising:
a substrate having a well formed thereon; and
a thermoresponsive polymer disposed in the well, the thermoresponsive polymer having a flow constrictor disposed thereon to constrict the flow of a fluid through a tube;
wherein the thermoresponsive polymer has an expanded volume at a lower temperature and the flow constrictor is disposed in a first position to at least partially constrict the flow of the fluid through the tube, and wherein the thermoresponsive polymer has a contracted volume at a higher temperature and the flow constrictor is disposed in a second position to constrict the flow of the fluid through the tube to a lesser degree than when the flow constrictor is disposed in the first position.

6. An apparatus as claimed in claim 5, wherein the thermoresponsive polymer is in a gel form.

7. An apparatus as claimed in claim 5, wherein the thermoresponsive polymer comprises at least one of poly(N-isopropylacrylamide) or poly(N-vinylcaprolactam).

8. An apparatus as claimed in claim 5, wherein the lower temperature is less than a lower critical solution temperature of the thermoresponsive polymer and the higher temperature is greater than the lower critical solution temperature of the thermoresponsive polymer.

9. An apparatus, comprising:
a substrate having a well formed thereon; and
a thermoresponsive polymer disposed in the well, the thermoresponsive polymer being coupled to a cantilevered member at a point distal from a pivot of the cantilevered member;
wherein the thermoresponsive polymer has an expanded volume at a lower temperature and the cantilevered member is disposed in a first angular position about the pivot, and wherein the thermoresponsive polymer has a contracted volume at a higher temperature and the cantilevered member is disposed in a second angular position about the pivot.

10. An apparatus as claimed in claim 9, wherein the thermoresponsive polymer is in a gel form.

11. An apparatus as claimed in claim 9, wherein the thermoresponsive polymer comprises at least one of poly(N-isopropylacrylamide) or poly(N-vinylcaprolactam).

12. An apparatus as claimed in claim 9, wherein the lower temperature is less than a lower critical solution temperature of the thermoresponsive polymer and the higher temperature is greater than the lower critical solution temperature of the thermoresponsive polymer.

13. An apparatus, comprising:
a substrate having a well formed thereon; and
a thermoresponsive polymer disposed in the well, the thermoresponsive polymer having a mirror disposed thereon;
wherein the thermoresponsive polymer has an expanded volume at a lower temperature and the mirror is disposed in a position to reflect an optical signal, and wherein the thermoresponsive polymer has a contracted volume at a higher temperature and the mirror is disposed in a second position to not reflect the optical signal.

14. An apparatus as claimed in claim 13, wherein the thermoresponsive polymer is in a gel form.

15. An apparatus switch as claimed in claim 13, wherein the thermoresponsive polymer comprises at least one of poly(N-isopropylacrylamide) or poly(N-vinylcaprolactam).

16. An apparatus switch as claimed in claim 13, wherein the lower temperature is less than a lower critical solution temperature of the thermoresponsive polymer and the higher temperature is greater than the lower critical solution temperature of the thermoresponsive polymer.

17. A method, comprising:
applying a first temperature to a thermoresponsive polymer to cause the thermoresponsive polymer to be in an expanded volume state;
applying a second temperature to the thermoresponsive polymer to cause the thermoresponsive polymer to be in a contracted volume;
wherein the change of volume state of the thermoresponsive polymer between the expanded volume state and the contacted volume state controls operation of an actuator.

18. A method as claimed in claim 17, wherein the thermoresponsive polymer is in a gel form.

19. A method as claimed in claim 17, wherein the thermoresponsive polymer comprises at least one of poly(N-isopropylacrylamide) or poly(N-vinylcaprolactam).

20. A method as claimed in claim 17, wherein the first temperature is a lower temperature being less than a lower critical solution temperature of the thermoresponsive polymer and the second temperature is a higher temperature being greater than the lower critical solution temperature of the thermoresponsive polymer.

21. A method as claimed in claim 17, wherein operation of the actuator includes at least one of electrical switching, valve controlling, motor operating, or optical switching.

22. A method as claimed in claim 17, wherein said applying a second temperature includes heating the thermoresponsive polymer with a heating element.

23. An apparatus, comprising:
a fluid source containing a fluid to be administered to a mammal; and
a valve to couple a catheter to the mammal, wherein the valve operates to control the flow of fluid from the fluid source to the mammal, the valve comprising:
a substrate having a well formed thereon; and
a thermoresponsive polymer disposed in the well, the thermoresponsive polymer having a flow constrictor disposed thereon to constrict the flow of a fluid through a tube;
wherein the thermoresponsive polymer has an expanded volume at a lower temperature and the flow constrictor is disposed in a first position to at least partially constrict the flow of the fluid through the catheter, and wherein the thermoresponsive polymer has a contracted volume at a higher temperature and the flow constrictor is disposed in a second position to constrict the flow of the fluid through the catheter to a lesser degree than when the flow constrictor is disposed in the first position.

24. An apparatus as claimed in claim 23, wherein the thermoresponsive polymer is in a gel form.

25. An apparatus as claimed in claim 23, wherein the thermoresponsive polymer comprises at least one of poly(N-isopropylacrylamide) or poly(N-vinylcaprolactam).

26. An apparatus as claimed in claim 23, wherein the lower temperature is less than a lower critical solution temperature of the thermoresponsive polymer and the higher temperature is greater than the lower critical solution temperature of the thermoresponsive polymer.

27. An apparatus as claimed in claim 23, wherein a temperature of the mammal is provided to the valve as a feedback signal to effectuate control of the flow of the fluid through the catheter to the animal.

28. An apparatus as claimed in claim 23, wherein an biological value of the mammal is provided to the valve as a feedback signal converted to a thermal stimulus to effectuate control of the flow of the fluid through the catheter to the animal.

29. An apparatus as claimed in claim 23, wherein the valve is disposed on a body of the mammal.

30. An apparatus as claimed in claim 23, wherein the valve is disposed within a body of the mammal.
